(19) United States
(12) Patent Application Publication
(10) Pub. No.: US 2009/0168050 A1
(43) Pub. Date: Jul. 2, 2009

(54) Optical Sensor System And Method

(75) Inventors: Don Nelson, Sam Ramon, CA (US); Robert W. Flager, Pleasanton, CA (US)

Correspondence Address: NELLCOR PURITAN BENNETT LLC;ATTN: IP LEGAL, 60 Middletown Avenue, North Haven, CT, 06473, US

(73) Assignee: NELLCOR PURITAN BENNETT LLC, Boulder, CO (US)

(21) Appl. No.: 12/343,770

(22) Filed: Dec. 24, 2008

Related U.S. Application Data
(60) Provisional application No. 61/009,333, filed on Dec. 27, 2007.

(51) Int. Cl. G01N 33/48

(52) U.S. Cl. 356/41

(57) ABSTRACT

The disclosed optical component may include an optical device and an overmold. The optical device may be configured to transmit or receive one or more wavelengths of light. The overmold may be disposed about the entirety of the optical device and may include a material transparent to the one or more wavelengths of light. A method of manufacturing a sensor may include overmolding an optical device with an overmold material that is transparent to a wavelength of light emitted or received by the optical device. The method may also include disposing the overmolded optical device proximate a sensor frame. The method may also include overmolding the sensor frame and the overmolded optical sensing device with a second overmold material. Further, the second overmold material may not block a portion of the overmolded optical device such that light can be emitted or received by the optical device without interference from the second overmold material.

### RELATED APPLICATIONS

This application claims priority to U.S. Provisional Application No. 61/009,333, filed Dec. 27, 2007, and is incorporated herein by reference in its entirety.

### BACKGROUND

The present disclosure relates generally to medical devices and, more particularly, to sensors used for sensing physiological parameters of a patient.

This section is intended to introduce the reader to various aspects of art that may be related to various aspects of the present invention, which are described and/or claimed below. This discussion is believed to be disclosure in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices and techniques have been developed for monitoring physiological characteristics. Such devices and techniques provide doctors and other healthcare personnel with the information they need to provide the best possible healthcare for their patients. As a result, these monitoring devices and techniques have become an indispensable part of modern medicine.

One such monitoring technique is commonly referred to as pulse oximetry. Pulse oximetry may be used to measure various blood flow characteristics, such as the blood-oxygen saturation of hemoglobin in arterial blood and/or the rate of blood pulsations corresponding to each heartbeat of a patient.

The devices based upon pulse oximetry techniques are commonly referred to as pulse oximeters. Pulse oximeters typically utilize a non-invasive sensor that is placed on or against a patient's tissue that is well perfused with blood, such as a patient's finger, toe, forehead or earlobe. The pulse oximeter sensor emits light and photoelectrically senses the absorption and/or scattering of the light after passage through the perfused tissue. The data collected by the sensor may be used to calculate one or more of the above physiological characteristics based upon the absorption or scattering of the light. More specifically, the emitted light is typically selected to be of one or more wavelengths that are absorbed or scattered in an amount related to the presence of oxygenated versus de-oxygenated hemoglobin in the blood. The amount of light absorbed and/or scattered may be used to estimate the amount of the oxygen in the tissue using various algorithms.

The sensors generally include an emitter that emits the light and a detector that detects the light. During use, the emitter and detector may be held against the patient's skin to facilitate the light being directed into and received from the skin of the patient. For example, a sensor may be clipped about a patients finger tip with the emitter placed on the finger nail, and the detector placed on the under side of the finger tip. When fitted to the patient, the emitted light may travel directly through the tissue of the finger and be detected without additional light being introduced or the emitted light being scattered. However, in practice, the shape and design of the sensor may not provide a tight fit between the sensor and the surface of the patient's skin and/or the sensor may be uncomfortable to the patient.

Further, during use, the emitter and the detector may be exposed to environmental conditions, such as condensation, liquids, debris and other substances that can degrade the performance of the sensor. For example, if the emitter or the detector is left exposed, substances, such as liquids, may migrate into the emitter and/or detector, potentially damaging electrical circuitry in the sensor and/or blocking the transmission of light.

### SUMMARY

Certain aspects commensurate in scope with the originally claimed invention are set forth below. It should be understood that these aspects are presented merely to provide the reader with a brief summary of certain forms any claimed invention might take and that these aspects are not intended to limit the scope of any claimed invention. Indeed, any claimed invention may encompass a variety of aspects that may not be set forth below.

In accordance with one aspect of the present disclosure, there may be provided an optical component. The optical component includes an optical device and an overmold. The optical device is configured to transmit or receive one or more wavelengths of light. The overmold is disposed about the entirety of the optical device, and comprises a material transparent to the one or more wavelengths of light.

In accordance with another aspect of the present disclosure, there may be provided and optical sensor system. The optical sensor system includes a frame, an optical device, a first overmold, and a second overmold. The first overmold encompasses the optical device, and is transparent to one or more wavelengths of light emitted by or received by the optical device. The second overmold encompasses the frame and does not cover at least a portion of the first overmold.

In accordance with another aspect of the present disclosure, there may be provided a method of manufacturing a sensor. The method includes overmolding an optical device with an overmold material that is transparent to a wavelength of light emitted or received by the optical device. The method also includes disposing the overmolded optical device proximate a sensor frame. Further, the method includes overmolding the sensor frame and the overmolded optical sensing device with a second overmold material. The second overmold material does not block a portion of the overmolded optical device such that light can be emitted or received by the optical device without interference from the second overmold material.

In accordance with yet another aspect of the present disclosure, there may be provided is pulse oximetry sensor. The pulse oximetry sensor includes a sensor frame, having a first frame portion and a second frame portion. The first frame portion having a first optical assembly. The first optical assembly includes an emitter configured to emit one or more wavelengths of light, an electrical or optical connection to the emitter, and a first overmold that encapsulates at least the emitter. Further, the first overmold comprises material transparent to the one or more wavelengths of light. The second frame portion includes a second optical assembly. The second optical assembly includes a photodetector configured to detect the one or more wavelengths of light, an electrical or optical connection to the photodetectors and a second overmold that encapsulates at least a portion of the photodetector. The second overmold comprises a material transparent to the one or more wavelengths of light. The pulse oximetry sensor also includes a third overmold that covers at least a portion of the sensor flame, and does not cover at least a portion of the first overmold or the second overmold.

### BRIEF DESCRIPTION OF THE DRAWINGS

The present disclosure may be better understood upon reading the following detailed description and upon reference to the drawings in which:

FIG. 1 illustrates a patient monitoring system coupled to a multi-parameter patient monitor and a sensor including an optical sensor, in accordance with aspects of the present disclosure;

FIG. 2A is a top view of a first embodiment of the optical sensor having a transparent overmold, in accordance with aspects of the present disclosure;

FIG. 2B is a side view of the first embodiment of the optical sensor having a transparent overmold, in accordance with aspects of the present disclosure;

FIG. 2C is an end view of the first embodiment of the optical sensor having a transparent overmold, in accordance with aspects of the present disclosure;

FIG. 3A is a top view of a second embodiment of the optical sensor having a transparent overmold, in accordance with aspects of the present disclosure;

FIG. 3B is a side view of the second embodiment of the optical sensor having a transparent overmold, in accordance with aspects of the present disclosure;

FIG. 3C is an end view of the second embodiment of the optical sensor having a transparent overmold, in accordance with aspects of the present disclosure;

FIG. 4 is a perspective view of an embodiment of the optical sensor including a surface feature, in accordance with aspects of the present disclosure;

FIG. 5 is a perspective view of an another embodiment of the optical sensor having a surface feature, in accordance with aspects of the present disclosure;

FIG. 6A is a top view of an embodiment of the optical sensor and a sensor frame, in accordance with aspects of the present disclosure;

FIG. 6B is a side view of the embodiment of the optical sensor and the sensor frame, in accordance with aspects of the present disclosure;

FIG. 6C is an end view of the embodiment of the optical sensor and the sensor frame, in accordance with aspects of the present disclosure;

FIG. 7A-7F illustrate a method of manufacturing the sensor, in accordance with aspects of the present disclosure; and

FIG. 8 is a side view of another embodiment of the sensor, in accordance with aspects of the present disclosure.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described herein, various embodiments of sensors are provided which are believed to provide a sealed sensor enclosure, good contact and comfortable fit for a range of patient anatomies, and a simplified method of manufacture. In general, embodiments of the sensors include optical components (e.g., emitters and detectors) that are overmolded with a transparent material that facilitates the passage of light to and from the optical components.

Further, in certain embodiments, the overmold material may include a flexible material that conforms to the shape of a patient finger, or other extremity, thereby providing a secure and comfortable fit the patient.

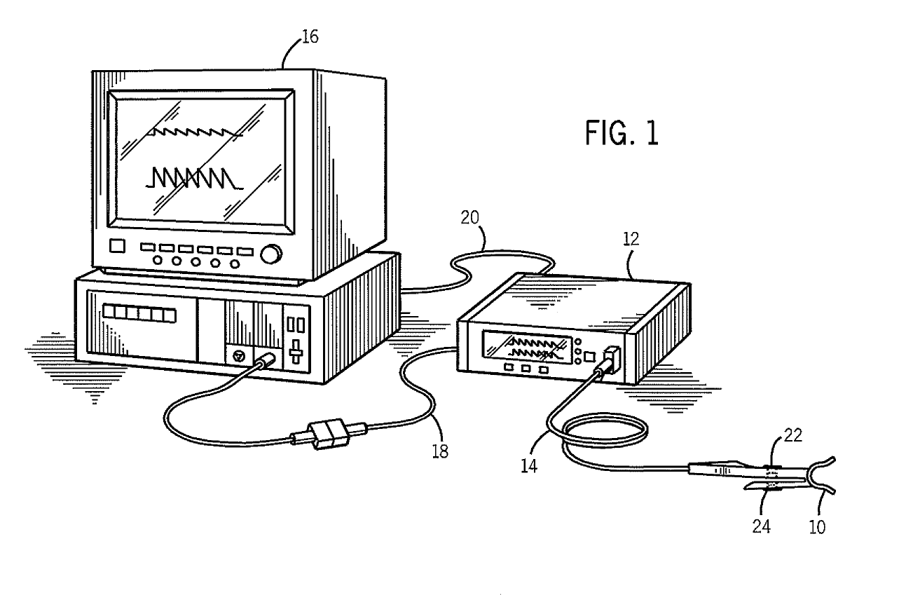

Prior to discussing such exemplary sensors in detail, it should be appreciated that such sensors are typically designed for use with a patient monitoring system. For example, referring now to FIG. 1, a sensor 10 may be used in conjunction with a patient monitor 12. In the depicted embodiment, a cable 14 connects the sensor 10 to the patient monitor 12. As will be appreciated by those of ordinary skill in the art, the sensor 10 and/or the cable 14 may include or incorporate one or more integrated circuit devices or electrical devices, such as a memory, processor chip, or resistor, that may facilitate or enhance communication between the sensor 10 and the patient monitor 12. Likewise the cable 14 may be an adaptor cable, with or without an integrated circuit or electrical device, for facilitating communication between the sensor 10 and various types of monitors, including older or newer versions of the patient monitor 12 or other physiological monitors. In other embodiments, the sensor 10 and the patient monitor 12 may communicate via wireless means, such as using radio, infrared, or optical signals. In such embodiments, a transmission device (not shown) may be connected to the sensor 10 to facilitate wireless transmission between the sensor 10 and the patient monitor 12. As will be appreciated by those of ordinary skill in the art, the cable 14 (or a corresponding wireless transmission) is typically used to transmit control or timing signals from the monitor 12 to the sensor 10 and/or to transmit acquired data from the sensor 10 to the monitor 12. In some embodiments, the cable 14 may be an optical fiber that enables optical signals to be conducted between the patient monitor 12 and the sensor 10.

In one embodiment, the patient monitor 12 may be a suitable pulse oximeter, such as those available from Nellcor Puritan Bennett LLC. In other embodiments, the patient monitor 12 may be a monitor suitable for measuring tissue water fractions, or other body fluid related metrics, using spectrophotometric or other techniques. Furthermore, the patient monitor 12 may be a multi-purpose monitor suitable for performing pulse oximetry and measurement of tissue water fraction, or other combinations of physiological and/or biochemical monitoring processes, using data acquired via the sensor 10. Furthermore, to upgrade conventional monitoring functions provided by the monitor 12 and to provide additional functions, the patient monitor 12 may be coupled to a multi-parameter patient monitor 16 via a cable 18 connected to a sensor input port and/or a cable 20 connected to a digital communication port.

The sensor 10, as depicted in FIG. 1, is a clip-style sensor that is overmolded to provide a unitary or enclosed assembly. The sensor 10 includes optical components, such as an emitter 22 and a detector 24, which may be of any suitable type. For example, the emitter 22 may be one or more light emitting diodes adapted to transmit one or more wavelengths of light, such as in the red to infrared range, and the detector 24 may be a photodetector, such as a silicon photodiode package, selected to receive light in the range emitted from the emitter 22. In the depicted embodiment, the sensor 10 is coupled to a cable 14 that is responsible for transmitting electrical and/or optical signals to and from the emitter 22 and the detector 24 of the sensor 10. The cable 14 may be permanently coupled to the sensor 10, or it may be removably coupled to the sensor 10—the latter alternative being more useful and cost efficient in situations where the sensor 10 is disposable.

The sensor 10 discussed herein may be configured for either transmission or reflectance type sensing. Furthermore, the sensor 10 may include various structural and functional features designed to facilitate its use. An example of such a sensor and its use and construction may be found in U.S. application Ser. No. 11/99,524 titled “Medical Sensor and Technique for Using the Same” and filed on Aug. 8, 2005, which is hereby incorporated by reference in its entirety for all purposes. As will be appreciated by those of ordinary skill in the art, however, such discussion is merely exemplary and is not intended to limit the scope of the present technique.

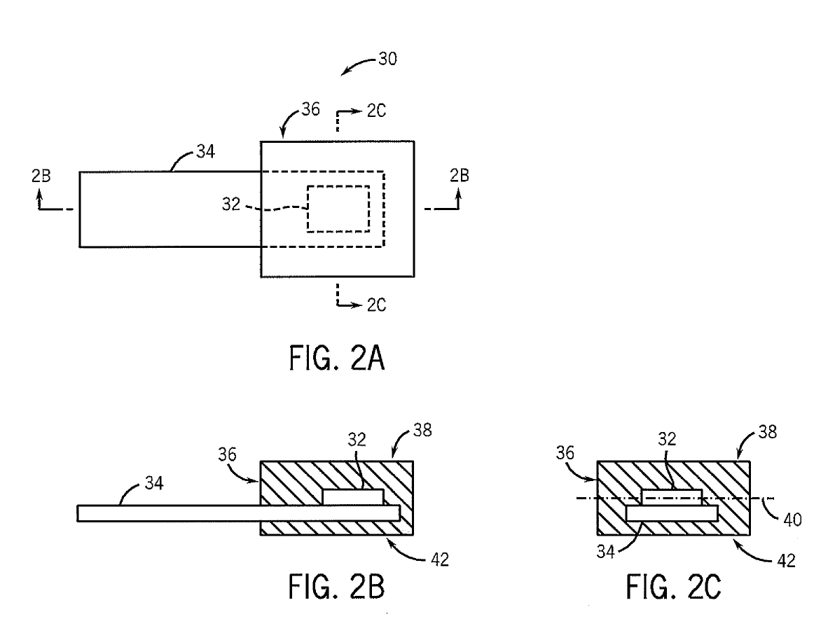

Turning now to FIG. 2A-2C, an embodiment of an optical assembly 30 is illustrated. As depicted, the optical assembly 30 may include an optical component 32 (such as an emitter 22 or a detector 24), a circuit 34, and a transparent overmold 36. The circuit 34 may include a flex circuit or other electrical connection that is configured to transmit signals between the optical component 32 and other components of the sensor 10, such as the cable 14. For example, in one embodiment, the circuit 34 may include a flexible substrate (e.g., a flex circuit) including a plurality of electrical traces that facilitate the transmission of power and other signals to the optical component 32. Accordingly, in an embodiment in which the optical component 32 includes the emitter 22, the circuit 34 may drive emission of one or more wavelengths emitted by the emitter 22, and in an embodiment in which the optical component 32 includes the receiver 24, the circuit 34 may transmit signals indicative of the light received by the receiver 24. Further, the circuit 34 may provide at least a minimal amount of structural support to the optical component 32. For example, the circuit 34 may include a semi-rigid structure that is capable of facilitating alignment of the optical component 32 in a mold or with respect to the sensor 10.

As illustrated, the overmold 36 may completely encompass the optical component 32 and encompass at least a portion of the circuit 34. For example, the overmold 36 may be formed around the optical component 32 such that it provides a complete seal/barrier between the optical component 32 and the surrounding environment. Thus, the overmold 36 may provide a hermetic seal around the optical component 32 that prevents, or at least reduces the likelihood of, substances (e.g., liquids and debris) from contacting and/or intruding on the optical component 32. Further, the overmold 36 may provide a similar hermetic seal around the portion of the circuit 34 that is encompassed by the overmold 36.

Further, the overmold 36 may facilitate deploying the sensor 10 in a variety of environments. For example, in an embodiment in which the overmold 36 encapsulates the optical component 32, the sensor 10 may be employed in environments where exposure to fluids (e.g., steam, water, saline solutions, blood and other medical fluids) is likely. Accordingly, the overmolded optics may facilitate various methods of cleaning and sterilization of the sensor 10. For example, in an embodiment in which the optical component 32 is hermetically sealed, the sensor 10 may be sterilized in an autoclave (e.g., a device that employs steam or other fluid at an elevated temperature and pressure) with a decreased risk of damaging the sensor 10.

In the illustrated embodiment, the overmold 36 includes a top face 38. During use of the sensor 10, the top face 38 may contact a patient's skin to facilitate the transmission of light between the optical assembly 30 and the patient's skin and tissue. As is discussed in further detail below, due to the transparent nature of the overmold 36 in certain embodiments, the top face 38 may define a window that enables light to pass to and from the optical sensor 32. For example, as depicted in the illustrated embodiment, the optical component 32 can be disposed internal to the overmold 36 such that it has a clear line of sight to and through the top face 38. In the depicted embodiment, the optical component 32 is disposed between the circuit 34 and the top face 38, and the optical component 32 is generally parallel the top face 38 such that the optical component 32 may emit or detect light via the top face 38. As will be appreciated, the use of the term “transparent” herein to describe the overmold 36 generally denotes that the overmold 36 freely passes the wavelengths of light emitted by the emitter 22 with little or no degradation or attenuation. The overmold 36, however, may or may not allow other wavelengths to be transmitted or may reduce or attenuate such other wavelengths.

In certain embodiments, the position of the optical component 32 may be modified to accommodate the optical characteristics of the optical component 32, as well as to provide comfort to the patient. For example, in the illustrated embodiment, the optical component 32 is located along a midline 40 of the overmold 36. The midline 40 includes a plane that extends through the overmold and that is approximately equal distance from the top face 38 and a bottom face 42 of the overmold 36. In one embodiment, the optical sensing device 40 may be positioned above the midline 40 (e.g., closer to the top face 38 than the bottom face 42) or below the midline 40 (e.g., closer to the bottom face 42 than the top face 38). Disposing the optical component 32 proximate the top face 42 may improve performance of the sensor by decreasing the distance the light travels through the overmold 36. Disposing the optical component 32 proximate the bottom face 42 may increase the comfort of the patient due to the increased amount of overmold material located between the top face 38 of the overmold 36 and the optical component 32. For example, in an embodiment in which the overmold 36 includes a soft (e.g., rubbery) material, as discussed in further detail below, the increased amount of overmold material between the top face 38 and the optical component 32 may facilitate the top face 38 of the overmold 36 conforming to the shape of the patient's finger or other extremity.

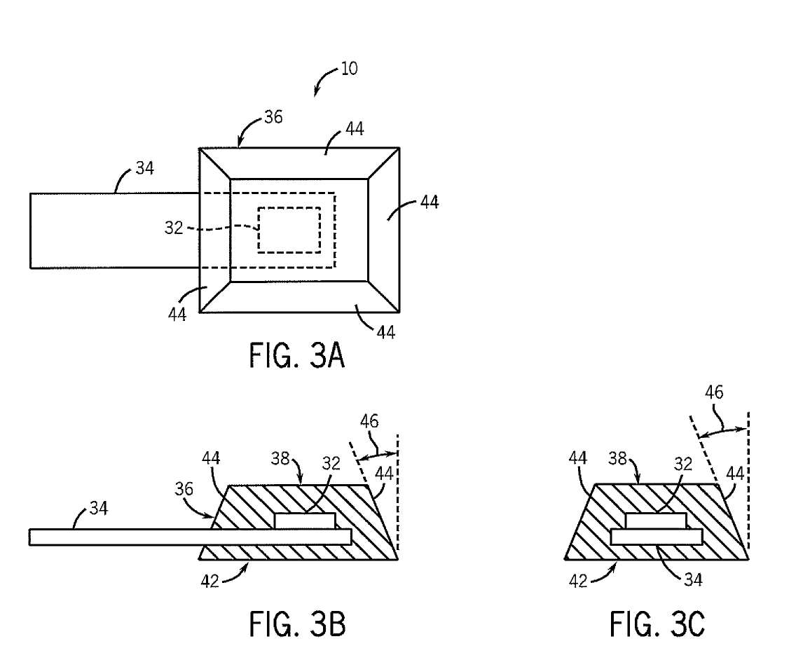

In the illustrated embodiment, the overmold 36 has a hexahedron shape. More specifically, the overmold 36 includes six faces that are generally orthogonal to one another (e.g., a cuboid or box-like shape). In other embodiments, the overmold 36 may include any variety of shapes conducive to a particular application. For example, as depicted in FIGS. 3A-3C, the overmold 36 may include at least one side that is tapered relative to top face 38 of the overmold 36. For example, in the illustrated embodiment, side faces 44 of the overmold 36 are tapered (i.e., oriented at an angle 46) such that the top face 38 has an area that is less than the area of the bottom face 42. In another embodiment, fewer than all of the side faces 44 may be oriented at the angle 46. For example, a single side face 44 may be oriented at an angle with the remaining three side faces 44 remaining orthogonal to one another. Further, the shape of the overmold 36 may take any form that provides for partially or fully encapsulating the optical component 32 while permitting light to pass to and from the optical component 32. For example, the overmold 36 may include a hemispherical shape, or any other polygon, having at least a portion of one face (e.g., the top face 38) that is configured to transmit light to and from the patient's skin and tissue.

As discussed in further detail below, the shape of the overmold 36 may also facilitate placement of the optical assembly 30 into a structure, such a frame of the sensor 10. For example, in one embodiment, the shape of the overmold 36 may be conducive to snapping the optical assembly 30 into a structure. In such an embodiment, the overmold 36 may provide an interference fit with the structure, or the overmold 36 may include the tapered side faces 44 that snap into the structure and are retained by complementary tapered faces and/or retention features of the structure.

In another embodiment, the shape of the overmold 36 may facilitate retention of the optical assembly 30 by an overmold that encapsulates at least a portion of the sensor 10. For example, as discussed in greater detain below, the overmolded optical assembly 30 may be disposed in the frame of the sensor 10, and the flame (including the optical assembly 30) subsequently overmolded to form the sensor 10. In such an embodiment, the tapered shape of the overmold 36 may prevent or reduce the likelihood of the optical assembly 30 dislodging from the sensor 10. Further, the shape of the overmold 36 may be varied in numerous configurations to facilitate retention in the sensor 10. For example, one or more of the side face 44 may include a concave shape, a convex shape, an indentation, a protrusion, or the like. Thus, when the frame of the sensor 10 is subsequently overmolded, the overmold may conform to and/or bond to the shape of the side faces 44 to prevent the optical assembly 30 from being dislodged from the sensor 10.

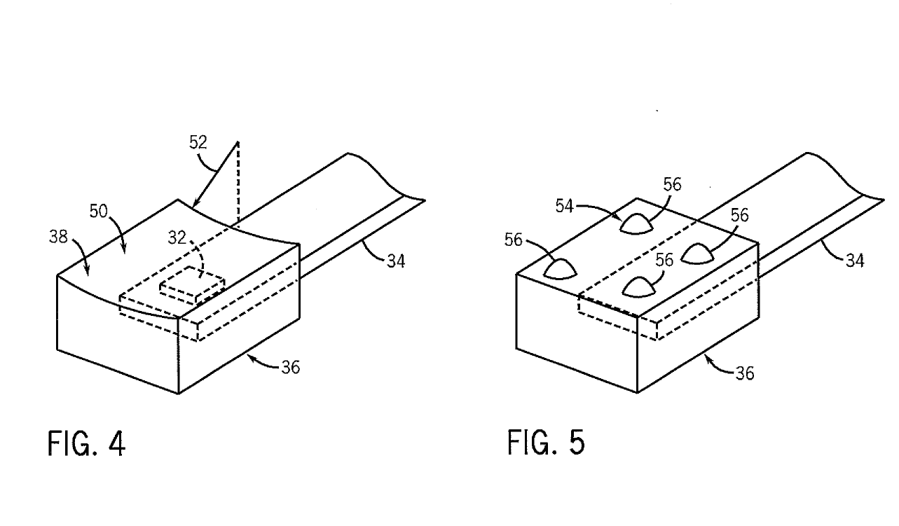

The overmold 36 may also include features that are conducive to promoting good and comfortable contact with the patient. As discussed previously, one embodiment may include disposing the optical component 32 proximate the bottom surface 42 of the overmold 36. Other embodiments may include providing surface features on the overmold 36 (e.g., features on the top face 38 of the overmold 36) that encourage good and comfortable contact between the patient and the sensor 10. For example, as depicted in the embodiment of FIG. 4, the top face 38 of the overmold 36 includes a curvature 50 having a radius 52. In the illustrated embodiment, the curvature 50 is concave, but may be convex, or a combination of convex and concave, in other embodiments. Further the shape (i.e., the radius 52) of the curvature 50 may complementary to a curvature of the location where the sensor 10 will be disposed (e.g., the patient's finger, finger nail, toe, forehead, or other extremity). Although the illustrated embodiment includes curvature across the width of the top face 38, the curvature 50 may include a variation along the length of the overmold 36, or along both the length and the width of the overmold 36 (e.g., forming a bow-like indentation).

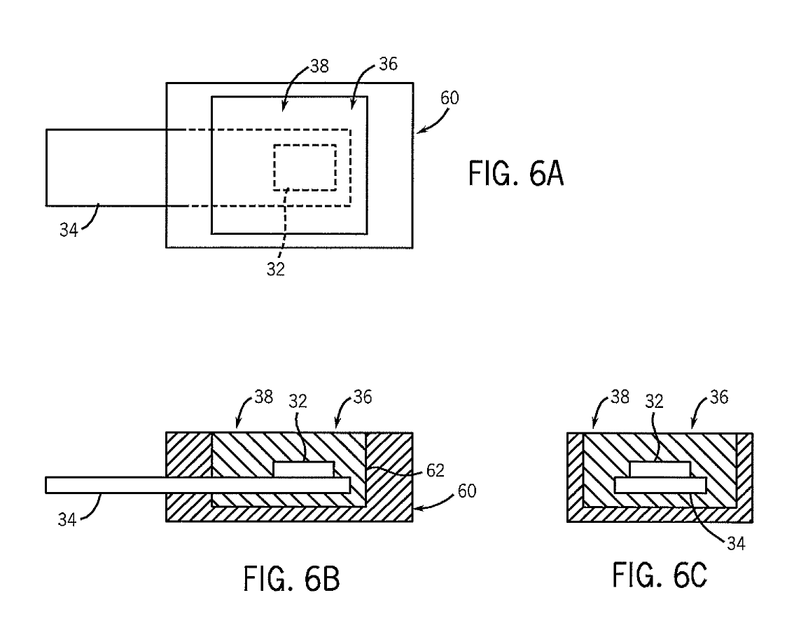

The curvature 50 may also promote efficient transfer of light through the overmold 36. In one embodiment, the radius of curvature 52 may be configured to focus the light in a given direction. For example, the curvature 50 (e.g., concave) of top face 38 may include a radius 52 that focuses the light emitted from the optical sensing device (e.g., the transmitter 22) onto the patient's skin and tissue. Similarly, the curvature 50 may include a shape that is configured to focus light onto the optical component 32 (e.g., the detector 24), in one embodiment. For example, the curvature 50 may include a convex shape that is configured to focus light from the top face 38 onto the embedded optical component 32. In other embodiments, the shape may be configured to scatter light. For example, the curvature may promote scattering the emitted light to increase the surface area impinged by the light. In another embodiment, the overmold 36 may include a surface texture that facilitates good contact with the patient. For example, as depicted in FIG. 5, the top face 38 of the overmold 36 may include texture features 54 that are configured to grip the patient's skin. As depicted, the texture features 54 may include protrusions 56 that prevent or reduce the likelihood of the sensor 10 sliding off of or otherwise moving relative to the patient. In the illustrated embodiment, the protrusions 56 include a plurality of bumps that extend out from the top face 38. In another embodiment, the surface features 54 may include other protrusions, such as ribs, ridges, or other raised areas on the top face 38 of the overmold 36. Similarly, the texture features 54 may include indentations in the top face 38 of the overmold 36. For example, in one embodiment, the top face 38 may include a plurality of dimples, troughs, cuts, or other depressions that are configured to grip the patient's skin.

The number and configuration of the surface texture features 54 may be varied in number, type and combination. For example, in the illustrated embodiment, the texture features 54 include four protrusions 56 disposed about the exterior of the top face 38 of the overmold 36. In another embodiment, any number and combination of the surface texture features 54 may be employed. For example, the top face 38 may include any number and any combination of protrusions or depressions.

In the illustrated embodiment, the area on the top face 38 that is directly above (e.g., in the line of sight of) the optical component 32 does not include surface texture features 54. The absence of surface texture features 54 may promote the efficient transfer of light through the overmold 38 by reducing the amount of light scattered at the top face 38. However, an embodiment may include surface texture features 54 in the region directly above the optical component 32. In such an embodiment, the surface texture features 54 may scatter the light passing through the overmold 36, such as to increase the surface area impinged by the light. In another embodiment, the surface texture features 54 may be employed to focus light toward the patient and/or to focus light toward the optical component 32. For example, as discussed previously a dimple or domed protrusion 56 may be located directly above the optical component 32 to focus emitted light into the skin of the patient or to focus light toward the optical component 32, respectively.

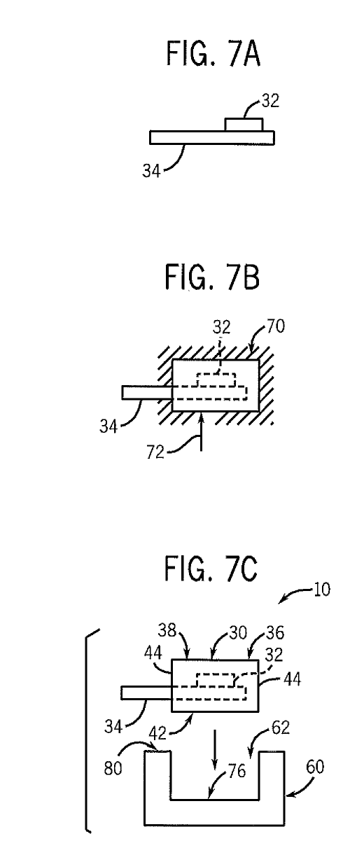

The optical assembly 30 (including the optical component 32, the circuit 34, and the overmold 36) may be assembled to the sensor 10 or similar supporting device, as discussed previously. For example, as depicted in FIG. 6, one embodiment may include disposing the optical assembly 30 into a flame 60 of the sensor 10. In the illustrated embodiment, the optical assembly 30 may be disposed into a cavity 62 of the frame 60.

In one embodiment, the optical assembly 30 may simply rest in the cavity 62. For example, the optical assembly 30 may be suspended into the cavity 62 without any significant restriction that couples the optical assembly 30 to the flame 60. However, in other embodiments, the optical assembly 30 may be secured to the cavity 62. For example, the optical assembly 30 may be secured to the cavity 62 via an interference fit, an adhesive, a mechanical fastener, and/or by subsequently overmolding the frame 60 after the optical assembly 30 has been disposed in the cavity 62.

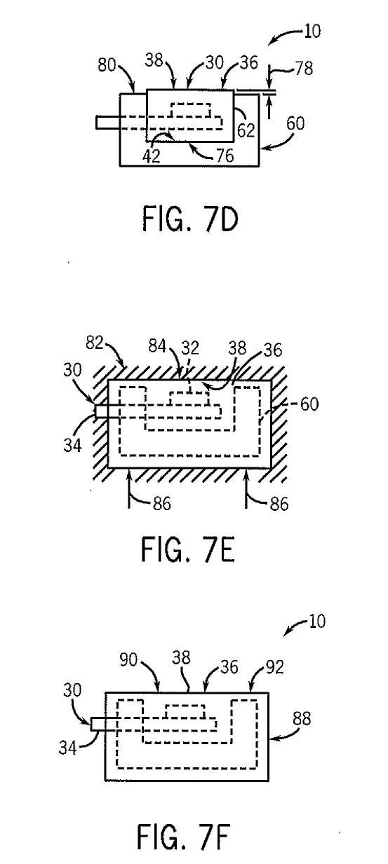

FIGS. 7A-7F are a series of illustrations that depict a method of manufacturing the sensor 10. FIG. 7A depicts providing the optical component 32 coupled to the circuit 34. For example, one embodiment may include providing the emitter 22 and/or the detector 24 electrically and/or mechanically coupled to the circuit (e.g., a flex circuit) 34, as discussed previously. FIG. 7B depicts inserting the optical assembly 30 into a mold 70. The mold 70 may include an injection mold or a casting mold, for instance. Once the optical assembly 30 is positioned in the mold 70, the material of the overmold 36 may be injected into the mold 70, as indicated by arrow 72. For example, as depicted, the optical assembly 30 maybe suspended in the mold 70 and the overmold material injected into a region between the optical assembly 30 and the walls of the mold 70 until the overmold material fills the mold 70 and encapsulates the optical assembly 30. In the illustrated embodiment, the optical component 32 may be completely encapsulated (i.e., surrounded on all six sides) by the overmold 36, and a portion of the circuit 34 proximate the optical component 32 may be at least partially encapsulated by the overmold 36.

After a given amount of time and/or once the overmold material has set, the optical assembly 30 may be removed from the mold 70. As illustrated in FIG. 7C, the optical assembly 30 (including the overmold 36) may then be positioned relative to the frame 60 of the sensor 10. As illustrated, the optical assembly 30 may be positioned in the cavity 62, as indicated by arrow 74. Positioning the optical assembly 30 into the cavity 62 may include mating the bottom face 42 of the overmold 36 to a bottom surface 76 of the cavity 62, as illustrated in the embodiment of FIG. 7D. Once fully inserted into the cavity 62, the top surface 38 of the overmold 36 may extend an offset distance 78 above a top surface 80 of the frame 60, as depicted in the illustrated embodiment. As discussed in further detail below, the offset distance 78 may enable a second overmold to be disposed over the top surface 80 of the frame 60 and flush with the top surface 38 of the optical assembly 30.

Once assembled, the optical assembly 30 and the frame 60 may be inserted into a second mold 82, as depicted in FIG. 7E. The top face 38 of the optical assembly 30 may be disposed in direct contact with an upper face 84 of the mold 82, in one embodiment. The upper face 84 of the second mold 82 may include a shape and/or texture that generally conforms to the shape of the top face 38 optical assembly 30. Thus, during the second overmold process, a second overmold material may enter the void regions of the second mold 82, but may not flow over and/or contact the top face 38 of the optical assembly 30. For example, in the illustrated embodiment, second overmold material may be injected into the second mold 82 as indicated by arrows 86, thereby filling the void region generally surrounding the optical assembly 30 and the frame 60 (including a region adjacent the top surface 80 of the frame 60) to form a sensor overmold 88. In one embodiment, the upper face 84 of the second mold 82 may include a material that is configured to conduct heat away from the optical assembly 30. For example, the upper face 84 may include beryllium copper or the like.

After a given amount of time and/or once the overmold material has set, the sensor 10 (including the optical assembly 30, the frame 60, and the overmold 88) may be removed from the second mold 82, as depicted in FIG. 7F. Subsequent to being removed, the sensor overmold 88 may encapsulate at least a substantial portion of the frame 60. At least a portion of the top surface 38 may not include the sensor overmold 88, thereby providing a window 90. The window 90 may facilitate the transmission of light to and from the optical component 32, as discussed previously. Further, an upper surface 92 of the sensor overmold 88 may be approximately flush with the top face 38 of the optical assembly 30. In one embodiment, the sensor overmold 88 does not extend over the top surface 38 of the optical assembly 30.

In the illustrated embodiment, the circuit 34 extends from the exterior of the sensor overmold 88. However, in one embodiment, the sensor overmold 88 may also encapsulate the circuit 34. For example, in one embodiment, the cable 14 may be coupled to the circuit 34 in the sensor overmold 88 and the cable 14 may extend external to the sensor overmold 88.

The method illustrated in FIGS. 7A-7F may be modified in various manners. For example, molding may include injection molding, casting, radiation curing, or similar methods of forming defined shapes in plastics. Further, in one embodiment, the offset 78 may be eliminated. In such an embodiment) the top surface 80 of the frame 60 and the top face 38 of the optical assembly 30 overmold 36 may both abut the top surface 84 of the second mold 83 such that the top surface 80 and the top face 38 are not overmolded. In another embodiment, during the molding process, the top face 38 and the top surface 80 of the frame 60 may not abut the top surface 84 of the mold 82, thus facilitating the overmold 88 encompassing the entirety of the optical assembly 30 and the frame 60. In such an embodiment, excess portions of the overmold 88 disposed on or over the top face 38 of the optical assembly 30 may be removed subsequently. For example, the overmold 88 may be milled, shaved, and/or dissolved with a solvent to expose the top face 38 of the optical assembly 30.

It is also noted that the sensor overmold 88 may also provide for securing the optical assembly 30 to the frame 60, as discussed previously. In one embodiment, the sensor overmold 88 may physically prevent the optical assembly 30 from dislodging from the frame 60. For example, material may overlap at least a portion of the top face 38 of the optical assembly 30. In another embodiment, the sensor overmold 88 may engage a taper of other feature of the side faces 44, thereby blocking the optical assembly 30 from dislodging from the frame 60. Further, in an embodiment, the overmold 88 may bond to at least some portion (e.g., the surface) of the optical assembly 30, thereby coupling the optical assembly 30 to the frame 30.

As illustrated in FIG. 7F, the overmold 88 may encompass a majority of the sensor 10 (including the frame 60 and the optical assembly 30), thereby providing an additional seal about the sensor 10. Similar to previous discussions, the overmold 88 may provide an additional level of hermetic sealing that is resistant to intrusion of liquids or other substances into the sensor 10.

As discussed previously, the overmold 36 may include a transparent material that is conducive to passing light through the top surface 38 (e.g., the window) of the optical assembly 30. In other words, the overmold 36 may include a material that facilitates the passage of light of at least a given wavelength (e.g., the wavelength transmitted or received by the emitter 22 and the detector 24) through the overmold 36, such that light may be emitted and or detected by the optical component 32 (e.g., the emitter 22 and the detector 24). In one embodiment, the overmold 36 may include a thermoplastic elastomer (TPE), styrene-butadiene (SBR), plasticized PVC, silicones, neoprene, isoprene, and other similar suitable materials, for example. The overmold material may include those manufactured by GLS Corporation, headquartered in McHenry, Ill., USA, and/or those manufactured by Teknor Apex Company, headquartered in Pawtucket, R.I., USA.

Further, the overmold 38 may include a soft and flexible material that is conducive to providing comfort to the patient at the interface between the patient and the optical assembly 30. In an embodiment, the material of the overmold 36 may conform to the shape of a patient's finger or other extremity, providing comfort and good contact (e.g., a minimal amount and number of gaps) between the top face 38 of the overmold 36 and the patient. For example, the overmold 38 may include a material having hardness between about 5 Shore A and about 90 Shore A. In one embodiment the overmold 36 may have a hardness below 60 Shore A.

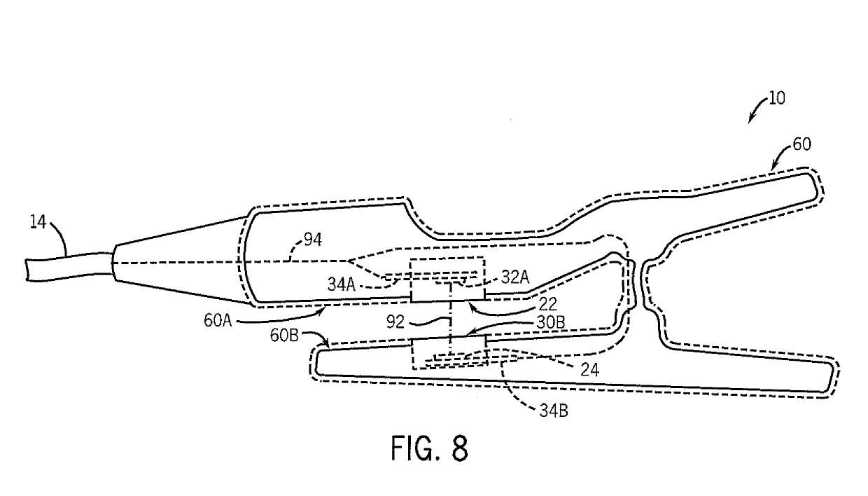

Turing now to FIG. 8, an embodiment of the sensor 10 is depicted. In the illustrated embodiment, the sensor 10 includes a first optical assembly 30A coupled to a first frame portion 60A, and a second optical assembly 30B coupled to a second frame portion 60B. Each of the optical sensors 30A and 30B include an overmold 36A and 36B that encapsulates an emitter 22 and a photodetector 24 and portions of circuits 34A and 34B, respectively. As depicted in the illustrated embodiment, the sensor 10 may include the overmold 88 encompassing substantially the all of the sensor 10, except at least a portion of the top faces 38A and 38B of the first and second optical assemblies 30A and 30B, respectively. In the illustrated embodiment, the sensor 10 includes additional circuitry 94 that electrically couples the circuits 34A and 34B to the cable 14, thereby facilitating the transmission of signals to and from the emitter 22 and the photodetector 24.

As discussed above, the portion of the top faces 38A and 38B that are not covered by the overmold 88 may provide a window for the passage of light between the emitter 22 and the photodetector 24. As illustrated, an embodiment may include the emitter 22 and the photodetector 24 directly opposing one another such that light may be transmitted (e.g., emitted and detected) along an axis 92 that is approximately normal to and extends between the emitter 22 and the photodetector 24. In use, the first frame portion 60A and the second frame portion 60B may be fit (e.g., clipped) around opposite sides of a patient finger, or other extremity, such that light can be emitted by the emitter 22 and received by the detector 24. Although the illustrated embodiment includes the emitter 22 in the first frame portion 60A and the photodetector 24 in the second frame portion 60B, the positions of the emitter 22 and the photodetector 24 may be arranged in various manners without changing the functionality of the sensor 10. For example, the positions of the emitter 22 and the photodetector 24 may be swapped.

While the medical sensors 10 discussed herein are some examples of integrally molded medical devices, other such devices are also contemplated and fall within the scope of the present disclosure. For example, other medical sensors and/or contacts applied externally to a patient may advantageously employ overmolded optical assemblies 30 including a transparent window. For example, devices for measuring tissue water fraction or other body fluid related metrics may utilize a sensor as described herein. Likewise, other spectrophotometric applications where a probe is attached to a patient may utilize a sensor as described herein.

While any claimed invention may be susceptible to various modifications and alternative forms, specific embodiments have been shown by way of example in the drawings and have been described in detail herein. However, it should be understood that any claimed invention is not intended to be limited to the particular forms disclosed. Rather, the invention is to cover all modifications, equivalents, and alternatives falling within the spirit and scope of the invention as defined by the following appended claims.

What is claimed is:

1. An optical component, comprising:
an optical device capable of transmitting or receiving one or more wavelengths of light; and
an overmold disposed generally about the entirety of the optical device, wherein the overmold comprises a material generally transparent to the one or more wavelengths of light.

2. The optical component of claim 1, wherein the overmold comprises a surface configured to contact a patient.

3. The optical component of claim 1, wherein the overmold comprises surface features configured to improve contact with a patient.

4. The optical component of claim 3, wherein the surface features comprise a curvature.

5. The optical component of claim 1, wherein the overmold comprises retention features configured to retain the overmold in a support structure.

6. The optical component of claim 5, wherein the retention features comprise tapered sides.

7. The optical component of claim 5, wherein the support structure comprises a sensor frame.

8. The optical component of claim 1, wherein the optical device comprises a light emitting diode.

9. The optical component of claim 1, wherein the optical device comprises a photodetector.

10. The optical component of claim 1, wherein the optical device is configured to be disposed in a spectrophotometric sensor.

11. The optical component of claim 1, wherein the overmold comprises a thermoplastic elastomer (TPE), styrene-butadiene (SBR), plasticized PVC, silicones, neoprene, and/or isoprene.

12. The optical component of claim 1, wherein the overmold has a hardness generally between about 5 Shore A and about 90 Shore A.

13. The optical component of claim 1, wherein the overmold has a hardness generally below about 60 Shore A.

14. An optical sensor system, comprising:
a frame;
an optical device disposed relative to the frame;
a first overmold that generally encompasses the optical device, wherein the first overmold is generally transparent to one or more wavelengths of light emitted by or received by the optical device; and
a second overmold that generally encompasses the frame, wherein the second overmold is configured not to cover at least a portion of the first overmold.

15. The optical sensor system of claim 14, wherein the first overmold comprises a first material comprising hardness between about 5 Shore A and about 90 Shore A.

16. The optical sensor system of claim 14, comprising a complementary optical device configured to receive light from or transmit light to the optical device, wherein the complementary optical device is overmolded with a material that is transparent to the one or more wavelengths of light.

17. The optical sensor system of claim 16, wherein the optical device is configured to transmit light comprising a first wavelength and the complementary optical device is configured to receive the light comprising the first wavelength.

18. The optical sensor system of claim 14, comprising a pulse oximetry monitor electrically coupled to the first optical sensing device.

19. A pulse oximetry sensor, comprising:
a sensor frame, comprising:
a first frame portion, comprising:
a first optical assembly, comprising:
an emitter capable of emitting one or more wavelengths of light; and
a first overmold that generally encapsulates at least the emitter, wherein the first overmold comprises material generally transparent to the one or more wavelengths of light; and
a second frame portion, comprising
a second optical assembly, comprising:
a photodetector configured to detect the one or more wavelengths of light; and
a second overmold that encapsulates at least a portion of the photodetector, wherein the second overmold comprises a material transparent to the one or more wavelengths of light; and
a third overmold that covers at least a portion of the sensor frame, and wherein the third overmold does not cover at least a portion of the first overmold or the second overmold.
